(19) United States
(12) Patent Application Publication
(10) Pub. No.: US 2009/0048181 A1
(43) Pub. Date: Feb. 19, 2009

(54) Combination therapy with glatiramer acetate and n-acetylcysteine for the treatment of multiple sclerosis

(76) Inventors: Hyman M. Schipper, Montreal (CA); Jean Godin, Montreal (CA)

Correspondence Address: COOPER & DUNHAM, LLP, 30 Rockefeller Plaza, 20th Floor, NEW YORK, NY, 10112, US

(21) Appl. No.: 11/661,060

(22) PCT Filed: Sep. 2, 2005
(86) PCT No.: PCT/US2005/031443

§ 371 Date: May 2, 2007

Related U.S. Application Data
(60) Provisional application No. 60/606,659, filed on Sep. 2, 2004.

(51) Int. Cl. A61K 38/07; A61P 37/00

(52) U.S. Cl. 514/18

(57) ABSTRACT

The subject invention provides a method of treating a subject afflicted with a form of multiple sclerosis comprising periodically administering to the subject an amount of glatiramer acetate and an amount of N-acetylcysteine or a pharmaceutically acceptable salt thereof, wherein the amounts when taken together are effective to alleviate a symptom of the form of multiple sclerosis in the subject so as to thereby treat the subject. Additionally, the subject invention provides a pharmaceutical composition comprising an amount of glatiramer acetate and an amount of N-acetylcysteine or a pharmaceutically acceptable salt thereof, wherein the amounts when taken together are effective to alleviate a symptom of a form of multiple sclerosis in a subject. The subject invention also provides a package comprising glatiramer acetate, N-acetylcysteine or a pharmaceutically acceptable salt thereof and instructions for use of the together to alleviate a symptom of a form of multiple sclerosis in a subject. The subject invention further provides a pharmaceutical combination comprising separate dosage forms of an amount of glatiramer acetate and an amount of N-acetylcysteine or a pharmaceutically acceptable salt thereof, which combination is useful to alleviate a symptom of a form of multiple sclerosis in a subject.

This application claims the benefit of U.S. Provisional Application No. 60/606,659, filed Sep. 2, 2004, the entire contents of which are hereby incorporated by reference.

Throughout this application, various publications are referenced in parenthesis. The disclosures of these publications in their entireties are hereby incorporated by reference into this application in order to more fully describe the state of the art to which this invention pertains.

### FIELD OF THE INVENTION

The subject invention relates to combination therapy for treating multiple sclerosis.

### BACKGROUND OF THE INVENTION

One of the more common chronic neurologic diseases in human adults is multiple sclerosis. This condition is a chronic, inflammatory CNS disease characterized pathologically by demyelination. There are five main forms of multiple sclerosis: 1) benign multiple sclerosis; 2) relapsing-remitting multiple sclerosis (RR-MS); 3) secondary progressive multiple sclerosis (SP-MS); 4) primary progressive multiple sclerosis (PP-MS); and 5) progressive-relapsing multiple sclerosis (PR-MS). Benign multiple sclerosis is a retrospective diagnosis which is characterized by 1-2 exacerbations with complete recovery, no lasting disability and no disease progression for 10-15 years after the initial onset. Benign multiple sclerosis may, however, progress into other forms of multiple sclerosis. Patients suffering from RR-MS experience sporadic exacerbations or relapses, as well as periods of remission. Lesions and evidence of axonal loss may or may not be visible on MRI for patients with RR-MS. SP-MS may evolve from RR-MS. Patients afflicted with SP-MS have relapses, a diminishing degree of recovery during remissions, less frequent remissions and more pronounced neurological deficits than RR-MS patients. Enlarged ventricles, which are markers for atrophy of the corpus callosum, midline center and spinal cord, are visible on MRI of patients with SP-MS. PP-MS is characterized by a steady progression of increasing neurological deficits without distinct attacks or remissions. Cerebral lesions, diffuse spinal cord damage and evidence of axonal loss are evident on the MRI of patients with PP-MS. PR-MS has periods of acute exacerbations while proceeding along a course of increasing neurological deficits without remissions. Lesions are evident on MRI of patients suffering from PR-MS (Multiple sclerosis: its diagnosis, symptoms, types and stages, 2003<http://www.albany.net/˜tjc/multiple-sclerosis.html>).

Researchers have hypothesized that multiple sclerosis is an autoimmune disease (Compston, Genetic susceptibility to multiple sclerosis, in McAlpine's Mutiple Sclerosis, Matthews, B. ed., London: Churchill Livingstone, 1991, 301-319; Hafler and Weiner, M S: A CNS and systemic autoimmune disease, Immunol. Today, 1989, 10:104-107; Olsson, Immunology of multiple sclerosis, Curr. Opin. Neurol. Neurosurg., 1992, 5:195-202). An autoimmune hypothesis is supported by the experimental allergic encephalomyelitis (EAE) model of multiple sclerosis, where the injection of certain myelin components into genetically susceptible animals leads to T cell-mediated CNS demyelination (Parkman, Graft-versus-host Disease, Ann. Rev. Med., 1991, 42: 189-197). Another theory regarding the pathogenesis of multiple sclerosis is that a virus, bacteria or other agent, precipitates an inflammatory response in the CNS, which leads to either direct or indirect (“bystander”) myelin destruction, potentially with an induced autoimmune component (Lampert, Autoimmune and virus-induced demyelinating diseases. A review, Am. J. Path., 1978, 91:176-208; Martyn, The epidemiology of multiple sclerosis, in McAlpine's Multiple Sclerosis, Matthews, B., ed., London: Churchil Livingstone, 1991, 3-40). Another experimental model of multiple sclerosis, Theiler's murine encephalomyelitis virus (TMEV)(Dal Canto, M. C., and H. L. Lipton. 1977., Multiple sclerosis. Animal model: Theiler's virus infection in mice, Am. J. Path. 88:497-500; Rodriguez, M. et al., Theiler's murine encephalomyelitis: a model of demyelination and persistence of virus. Crit. Rev. Immunol., 1987, 7:325), supports the theory that a foreign agent initiates multiple sclerosis. In the TMEV model, injection of the virus results in spinal cord demyelination.

Glatiramer acetate (GA), also known as Copolymer-1, has been shown to be effective in treating multiple sclerosis (MS) (Lampert, see above). Daily subcutaneous injections of glatiramer acetate (20 mg/injection) reduce relapse rates, progression of disability, appearance of new lesions by magnetic resonance imaging (MRI), (Johnson et al., Copolymer 1 reduces relapse rate and improves disability in relapsing-remitting multiple sclerosis: results of a phase III multicenter, double-blind placebo-controlled trial, The Copolymer 1 Multiple Sclerosis Study Group, Neurol., 1995, 45:1268) and appearance of “black holes” (Filippi et al., Glatiramer acetate reduces the proportion of MS lesions evolving into black holes, Neurol., 2001, 57:731-733).

COPAXONE® is the brand name for a formulation containing glatiramer acetate as the active ingredient. Glatiramer acetate is approved for reducing the frequency of relapses in relapsing-remitting multiple sclerosis. Glatiramer acetate consists of the acetate salts of synthetic polypeptides containing four naturally occurring amino acids: L-glutamic acid, L-alanine, L-tyrosine, and L-lysine with an average molar fraction in COPAXONE® of 0.141, 0.427, 0.095 and 0.338, respectively. In COPAXONE®, the average molecular weight of the glatiramer acetate is 4,700-11,000 daltons. Chemically, glatiramer acetate is designated L-glutamic acid polymer with L-alanine, L-lysine and L-tyrosine, acetate (salt). Its structural formula is:

(Glu,Ala,Lys,Tyr)ₓ.CH₃COOH (C₅H₉NO₄.C₃H₇NO₂.C₆H₁₄N₂O₂.C₉H₁₁NO₃)ₓ.χC₂H₄O₂
CAS-147245-92-9.

The recommended dosing schedule of COPAXONE® for relapsing-remitting multiple sclerosis is 20 mg per day injected subcutaneously. COPAXONE® Injection is a clear colorless to slightly yellow, sterile, non-pyrogenic solution for subcutaneous injection. Each 1.0 mL of solution contains 20 mg of glatiramer acetate and 40 mg of mannitol, USP. The pH range of the solution is approximately 5.5 to 8.5, (“COPAXONE®” in Physician's Desk Reference, Medical Economics Co., Inc., Montvale, N.J., 2003, 3214-3218; see also U.S. Pat. Nos. 3,849,550; 5,800,808; 5,858,964, 5,981,589; 6,048,898; 6,054,430; 6,214,791; 6,342,476; and 6,362,161, all of which are hereby incorporated by reference).

Lehmann disclosed that the administration of N-acetylcysteine (NAC) to SJL/J mice attenuated the induction of EAE, a murine model of multiple sclerosis, and in animals with established EAE, improvement correlated with N-acetylcysteine treatment in a dose-related manner (Lehmann et al., Oral administration of the oxidant-scavenger N-acetyl-L-cysteine inhibits acute experimental autoimmune encephalomyelitis, J. Neuroimmunol., 1994, 50: 35-42). Others have suggested that N-acetylcysteine may be beneficial in the treatment of multiple sclerosis in humans, but have not tested N-acetylcysteine in humans (HSI Healing Network Leads to Breakthrough Against Multiple Sclerosis, Health Sciences Institute, 1999, <http://www.cnm-inc.com/HSI_Multiple_Sclerosis>; Rothfeld, G., Supplement Recommendations for Multiple Sclerosis <http://www.wholehealthmd.com/print/view/1,1560,RA_{—}493_supp,00.html>; Wilder, Free Radicals and Neuroprotection, The ChiropracticResourceOrganization<http://www.chiro.org/nutritit ion/ABSTRACTS/Neuroprotect.shtml>). Wilder tested N-acetylcysteine in ten human patients suffering from multiple sclerosis and found an elevation of TNF-α, but were unable to ascertain the efficacy of N-acetylcysteine in the treatment of multiple sclerosis in their preliminary studies (Wilder et al., Treatment of Neurodegenerative Disease with N-acetylcysteine, First Annual Health Science Center Research Week, Oct. 16-20, 1995<http://neuro-www.mgh.harvard.edu/neurowebforum/Multiple SclerosisArticles/NACReport>).

N-acetylcysteine is commercially available under the tradename, SOLMUCOL® Parvolex® and MUCOMYST®. N-acetylcysteine is indicated as adjunctive therapy in respiratory conditions and to reduce the viscosity of mucus associated with cystic fibrosis. N-acetylcysteine is also effective against paracetamol and acetaminophen overdosages. The recommended dose for the treatment of respiratory disorders is as follows: adults—200 mg every 8 hours; children 2-6 years of age—200 mg every 12 hours; and children 2 years of age and under—200 mg daily. For mucolytic therapy in cystic fibrosis, the recommended dose is as follows: adults—200-400 mg every 8 hours; and children 2-6 years of age—200 mg every 8 hours. For paracetamol overdosage, the recommended dosage for patients of all ages is 140 mg/kg followed by 70 mg/kg every 4 hours for 17 doses (SOLMUCOL® 200 and SOLMUCOL® granules for solution, SOLMUCOL® lozenges, Lagamed Package Insert <http://www.home.intekom.com/pharm/lagamed/solmucol.html>).

The administration of two drugs to treat a given condition, such as a form of multiple sclerosis, raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (Guidance for Industry, In vivo drug metabolism/drug interaction studies—study design, data analysis, and recommendations for dosing and labeling, U.S. Dept. Health and Human Svcs., FDA, Ctr. for Drug Eval. and Res., Ctr. for Biologics Eval. and Res., Clin./Pharm., November 1999<http://www.fda.gov/cber/gdlns/metabol.pdf>). Thus, when two drugs are administered to treat the same condition, it is unpredictable whether each will complement, have no effect on, or interfere with, the therapeutic activity of the other in a human subject.

Not only may the interaction between two drugs affect the intended therapeutic activity of each drug, but the interaction may increase the levels of toxic metabolites (Guidance for Industry, In vivo drug metabolism/drug interaction studies—study design, data analysis, and recommendations for dosing and labeling, U.S. Dept. Health and Human Svcs., FDA, Ctr. for Drug Eval. and Res., Ctr. for Biologics Eval. and Res., Clin./Pharm., November 1999<http://www.fda.gov/cber/gdlns/metabol.pdf>). The interaction may also heighten or lessen the side effects of each drug. Hence, upon administration of two drugs to treat a disease, it is unpredictable what change will occur in the negative side profile of each drug.

Additionally, it is difficult to accurately predict when the effects of the interaction between the two drugs will become manifest. For example, metabolic interactions between drugs may become apparent upon the initial administration of the second drug, after the two have reached a steady-state concentration or upon discontinuation of one of the drugs (Guidance for Industry, In vivo drug metabolism/drug interaction studies—study design, data analysis, and recommendations for dosing and labeling, U.S. Dept. Health and Human Svcs., FDA, Ctr. for Drug Eval. and Res., Ctr. for Biologics Eval. and Res., Clin./Pharm., November 1999<http://www.fda.gov/cber/gdlns/metabol.pdf>).

Thus, the success of one drug or each drug alone in an in vitro model, an animal model, or in humans, may not correlate into efficacy when both drugs are administered to humans.

In accordance with the subject invention, glatiramer acetate and N-acetylcysteine or a pharmaceutically acceptable salt thereof are effective in combination to treat a form of multiple sclerosis, specifically, relapsing-remitting multiple sclerosis.

### SUMMARY OF THE INVENTION

The subject invention provides a method of treating a subject afflicted with a form of multiple sclerosis comprising periodically administering to the subject an amount of glatiramer acetate and an amount of N-acetylcysteine or a pharmaceutically acceptable salt thereof, wherein the amounts when taken together are effective to alleviate a symptom of the form of multiple sclerosis in the subject so as to thereby treat the subject.

<See pages 11-12 below for refinement of the term “periodically”.>

The subject invention further provides a pharmaceutical composition comprising an amount of glatiramer acetate and an amount of N-acetylcysteine or a pharmaceutically acceptable salt thereof, wherein the amounts when taken together are effective to alleviate a symptom of a form of multiple sclerosis in a subject.

In addition, the subject invention provides a package comprising
i) a first pharmaceutical composition comprising an amount of glatiramer acetate and a pharmaceutically acceptable carrier;
ii) a second pharmaceutical composition comprising an amount of N-acetylcysteine or a pharmaceutically acceptable salt thereof and a pharmaceutically acceptable carrier; and
iii) instructions for use of the first and second pharmaceutical compositions together to alleviate a symptom of a form of multiple sclerosis in a subject.

### DETAILED DESCRIPTION OF THE INVENTION

The subject invention provides a method of treating a subject afflicted with a form of multiple sclerosis comprising periodically administering to the subject an amount of glatiramer acetate and an amount of N-acetylcysteine or a pharmaceutically acceptable salt thereof, wherein the amounts when taken together are effective to alleviate a symptom of the form of multiple sclerosis in the subject so as to thereby treat the subject.

The pharmaceutically acceptable salt of N-acetylcysteine may be any pharmaceutically acceptable salt (B Sepacia Problems Remain, Cystic Fibrosis Currents, Spring 1998 Ortho-McNeil CF Care <http://www.cfcare.com/news/currents/jan98/page6.html>), such as those disclosed by Remington (Remington, et al., The Science and Practice of Pharmacy, 20^{th} ed., A. Gennaro et al., eds., Lippincott Williams and Wilkins, Philadelphia, Pa., 2000, 704-712). In one embodiment, the pharmaceutically acceptable salt of N-acetylcysteine is the lysine salt (Nacystelyn, a novel lysine salt of N-acetylcysteine, to augment cellular antioxidant defence in vitro, Respir. Med. (England), 1997, 91(3): 159-168<http://www.lef.org/protocols/abstracts/abstr-096.html>).

In one embodiment, the form of multiple sclerosis is relapsing-remitting multiple sclerosis.

In another embodiment, the subject is a human being.

In a further embodiment, each of the amount of glatiramer acetate when taken alone, and the amount of N-acetylcysteine or the pharmaceutically acceptable salt thereof when taken alone is effective to alleviate the symptom of the form of multiple sclerosis.

In an embodiment, either the amount of glatiramer acetate when taken alone, the amount of N-acetylcysteine or the pharmaceutically acceptable salt thereof when taken alone or each such amount when taken alone is not effective to alleviate the symptom of the form of multiple sclerosis.

In yet another embodiment, the symptom is the frequency of relapses, the frequency of clinical exacerbation, or the accumulation of physical disability.

In one embodiment, the amount of glatiramer acetate may be 10 to 80 mg; or 12 to 70 mg; or 14 to 60 mg; or 16 to 50 mg; or 18 to 40 mg; or 20 to 30 mg; or 20 mg. For each amount of glatiramer acetate, the amount of N-acetylcysteine or the pharmaceutically acceptable salt thereof may be 500 mg-20 g; or 750 mg-15 g; or 1-10 g; or 3-8 g; or 4-6 g; or 5 g. In an alternative, the amount of N-acetylcysteine or the pharmaceutically acceptable salt thereof may be 4 g or 6 g.

Alternatively, the amount of N-acetylcysteine or the pharmaceutically acceptable salt thereof is in the range of 750 mg-15 g/day.

In another embodiment, the amount of N-acetylcysteine or the pharmaceutically acceptable salt thereof is in the range of 1-10 g/day.

In another embodiment, the amount of N-acetylcysteine or the pharmaceutically acceptable salt thereof is in the range of 3-8 g/day.

In another embodiment, the amount of N-acetylcysteine or the pharmaceutically acceptable salt thereof is in the range of 4-6 g/day.

In another embodiment, the amount of N-acetylcysteine or the pharmaceutically acceptable salt thereof is 4 g/day.

In another embodiment, the amount of N-acetylcysteine or the pharmaceutically acceptable salt thereof is 6 g/day.

Alternatively, the amount of glatiramer acetate may be in the range from 10 to 600 mg/week; or 100 to 550 mg/week; or 150 to 500 mg/week; or 200 to 450 mg/week; or 250 to 400 mg/week; or 300 to 350 mg/week; or 300 mg/week.

In another embodiment, the amount of glatiramer acetate may be in the range from 50 to 150 mg/day; or 60 to 140 mg/day; or 70 to 130 mg/day; or 80 to 120 mg/day; or 90 to 110 mg/day; or 100 mg/day.

Alternatively, the amount of glatiramer acetate may be in the range from 10 to 80 mg/day; or 12 to 70 mg/day; or 14 to 60 mg/day; or 16 to 50 mg/day; or 18 to 40 mg/day; or 19 to 30 mg/day; or 20 mg/day.

In one embodiment, the glatiramer acetate is in solution with 40 mg of manitol, USP.

In one embodiment, the periodic administration of glatiramer acetate is effected daily.

In another embodiment, the periodic administration of glatiramer acetate is effected twice daily at one half the amount.

In another embodiment, the periodic administration of N-acetylcysteine or the pharmaceutically acceptable salt thereof is effected twice daily at one half the amount.

In an additional embodiment, the periodic administration of glatiramer acetate is effected once every 3 to 11 days; or once every 5 to 9 days; or once every 7 days; or once every 24 hours.

For each administration schedule of glatiramer acetate, the N-acetylcysteine or the pharmaceutically acceptable salt thereof may be administered once every 4-12 hours; or once every 6-10 hours; or once every 8 hours or twice daily.

In a further embodiment, the administration of the glatiramer acetate substantially precedes the administration of the N-acetylcysteine or the pharmaceutically acceptable salt thereof.

In an added embodiment, the administration of the N-acetylcysteine or the pharmaceutically acceptable salt thereof substantially precedes the administration of the glatiramer acetate.

In one embodiment, the glatiramer acetate and the N-acetylcysteine or the pharmaceutically acceptable salt thereof may be administered for a period of time of at least 4 days. In a further embodiment, the period of time may be 5 days to 5 years; or 10 days to 3 years; or 2 weeks to 1 year; or 1 month to 6 months; or 3 months to 4 months. In yet another embodiment, the glatiramer acetate and the N-acetylcysteine or the pharmaceutically acceptable salt thereof may be administered for the lifetime of the subject.

The administration of glatiramer acetate or N-acetylcysteine or the pharmaceutically acceptable salt thereof may each independently be oral, nasal, pulmonary, parenteral, intravenous, intra-articular, transdermal, intradermal, subcutaneous, topical, intramuscular, rectal, intrathecal, intraocular, buccal or by gavage. For N-acetylcysteine or the pharmaceutically acceptable salt thereof, the preferred route of administration is oral or by gavage. The preferred route of administration for glatiramer acetate is subcutaneous or oral. One of skill in the art would recognize that doses at the higher end of the range may be required for oral administration.

In one embodiment, the administration of the glatiramer acetate may be subcutaneous, intraperitoneal, intravenous, intramuscular, intraocular or oral and the administration of the N-acetylcysteine or the pharmaceutically acceptable salt thereof may be oral. In another embodiment, the administration of the glatiramer acetate may be subcutaneous and the administration of the N-acetylcysteine may be oral.

The subject invention further provides a pharmaceutical composition comprising an amount of glatiramer acetate and an amount of N-acetylcysteine or a pharmaceutically acceptable salt thereof, wherein the amounts when taken together are effective to alleviate a symptom of a form of multiple sclerosis in a subject.

In one embodiment of the pharmaceutical composition, each of the amount of glatiramer acetate when taken alone and the amount of N-acetylcysteine or the pharmaceutically acceptable salt thereof when taken alone is effective to alleviate the symptom of multiple sclerosis.

In another embodiment of the pharmaceutical composition, either of the amount of glatiramer acetate when taken alone, or the amount of N-acetylcysteine or the pharmaceutically acceptable salt thereof when taken alone or each such amount when taken alone is not effective to alleviate the symptom of multiple sclerosis.

In one embodiment of the pharmaceutical composition, the amount of glatiramer acetate may be in the range from 10 to 600 mg; or 100 to 550 mg; or 150 to 500 mg; or 200 to 450 mg; or 250 to 400 mg; or 300 to 350 mg; or 300 mg.

In a further embodiment of the pharmaceutical composition, the amount of glatiramer acetate may be in the range from 10 to 80 mg; or 12 to 70 mg; or 14 to 60 mg; or 16 to 50 mg; or 18 to 40 mg; or 19 to 30 mg; or 20 mg.

Alternatively, the amount of glatiramer acetate in the pharmaceutical composition may be in the range from 50 to 150 mg; or 60 to 140 mg; or 70 to 130 mg; or 80 to 120 mg; or 90 to 110 mg; or 100 mg.

For each amount of glatiramer acetate in the pharmaceutical composition, the amount of N-acetylcysteine or the pharmaceutically acceptable salt thereof in the pharmaceutical composition may be 500 mg-20 g; or 750 mg-15 g; or 1-10 g; or 3-8 g; or 4-6 g; or 5 g.

The subject invention also provides a package comprising
i) a first pharmaceutical composition comprising an amount of glatiramer acetate and a pharmaceutically acceptable carrier;
ii) a second pharmaceutical composition comprising an amount of N-acetylcysteine or a pharmaceutically acceptable salt thereof and a pharmaceutically acceptable carrier; and
iii) instructions for use of the first and second pharmaceutical compositions together to alleviate a symptom of a form of multiple sclerosis in a subject.

In an embodiment of the package, the amount of glatiramer acetate may be in the range from 10 to 600 mg; or 100 to 550 mg; or 150 to 500 mg; or 200 to 450 mg; or 250 to 400 mg; or 300 to 350 mg; or 300 mg.

In another embodiment of the package, the amount of glatiramer acetate may be in the range from 10 to 80 mg; or 12 to 70 mg; or 14 to 60 mg; or 16 to 50 mg; or 18 to 40 mg; or 19 to 30 mg; or 20 mg.

Alternatively, the amount of glatiramer acetate in the package may be in the range from 50 to 150 mg; or 60 to 140 mg; or 70 to 130 mg; or 80 to 120 mg; or 90 to 110 mg; or 100 mg.

For each amount of glatiramer acetate in the package, the amount of N-acetylcysteine or the pharmaceutically acceptable salt thereof in the package may be 500 mg-20 g; or 750 mg-15 g; or 1-10 g; or 3-8 g; or 4-6 g; or 5 g.

In an embodiment, the amount of N-acetylcysteine or the pharmaceutically acceptable salt thereof in the package may be in multiple 2.5 g doses, e.g. two 2.5 g does.

The subject invention further provides a pharmaceutical combination comprising separate dosage forms of an amount of glatiramer acetate and an amount of N-acetylcysteine or a pharmaceutically acceptable salt thereof, which combination is useful to alleviate a symptom of a form of multiple sclerosis in a subject.

In an embodiment of the pharmaceutical combination, each of the amount of glatiramer acetate when taken alone and the amount of N-acetylcysteine or the pharmaceutically acceptable salt thereof when taken alone is effective to alleviate the symptom of multiple sclerosis.

In an additional embodiment of the pharmaceutical combination, either of the amount of glatiramer acetate when taken alone, the amount of N-acetylcysteine or the pharmaceutically acceptable salt thereof when taken alone or each such amount when taken alone is not effective to alleviate the symptom of multiple sclerosis.

In a further embodiment, the pharmaceutical combination may be for simultaneous, separate or sequential use to treat the form of multiple sclerosis in the subject.

The subject invention further provides a product containing glatiramer acetate and N-acetylcysteine or the pharmaceutically acceptable salt thereof as a combined preparation for simultaneous, separate or sequential use in a therapy.

An additional embodiment of the product may be for use in a therapy for multiple sclerosis.

The subject invention is also a use of glatiramer acetate and N-acetylcysteine or the pharmaceutically acceptable salt thereof for the manufacture of a combined preparation medicament of the treatment of multiple sclerosis, wherein glatiramer acetate and N-acetylcysteine or the pharmaceutically acceptable salt thereof are to be administered simultaneously, separately or sequentially.

The subject application further provides a package containing glatiramer acetate and instructions for the simultaneous, separate or sequential administration with glatiramer acetate of N-acetylcysteine or the pharmaceutically acceptable salt thereof.

In an embodiment, the package is for use in treating multiple sclerosis.

Formulations of the invention suitable for oral administration may be in the form of capsules, pills, tablets, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of the active compound or compounds.

In solid dosage forms of the invention for oral administration (capsules, tablets, pills, dragees, powders, granules and the like), the active ingredient(s) is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, calcium phosphate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration of the active ingredients include pharmaceutically acceptable emulsions, microemulsions, solutions, suspensions, syrups and elixirs. In addition to the active ingredient(s), the liquid dosage forms may contain inert dilutents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to the active compounds, may contain suspending agents such as ethoxylated isostearyl alcohols, polyoxyethylene sorbitol and sorbitan esters, microcrystalline cellulose, aluminum metahydroxide, bentonite, agar-agar and tragacanth, and mixtures thereof.

The pharmaceutical compositions, particularly those comprising glatiramer acetate, may also include human adjuvants or carriers known to those skilled in the art. Such adjuvants include complete Freund's adjuvant and incomplete Freund's adjuvant. The compositions may also comprise wetting agents, emulsifying and suspending agents, sweetening, flavoring, coloring, perfuming and preservative agents.

Glatiramer acetate may be formulated into pharmaceutical compositions with pharmaceutically acceptable carriers, such as water or saline and may be formulated into eye drops. Glatiramer acetate may also be formulated into delivery systems, such as matrix systems.

This invention will be better understood from the Experimental Details which follow. However, one skilled in the art will readily appreciate that the specific methods and results discussed are merely illustrative of the invention as described more fully in the claims which follow thereafter.

### Experimental Details

### Clinical Trial of Relapsing-Remitting Multiple Sclerosis

The purpose of this trial is to evaluate the treatment of participants with relapsing-remitting multiple sclerosis (RR-MS) with COPAXONE® in combination with N-acetylcysteine or a pharmaceutically acceptable salt thereof. The clinical objective is to evaluate the effect of treatments on MRI variables, clinical evaluations and immunological profile.

The design of this trial is a open label one group study of COPAXONE® in combination with N-acetylcysteine or a pharmaceutically acceptable salt thereof for the treatment of relapsing-remitting multiple sclerosis. Eighteen (18) patients with RR-MS who meet the inclusion/exclusion criteria are enrolled in the study. Patients receive 20 mg SQ (subcutaneous) of COPAXONE® daily plus 5 g/day of N-acetylcysteine or a pharmaceutically acceptable salt thereof orally in two equal doses, i.e., 2.5 g twice per day.

Participant inclusion criteria are as follows: 1) Clinically definite multiple sclerosis (CDMS) as defined by Poser et al. (Ann. Neurol. 1983) with disease duration (from onset) of at least 6 months; 2) Subjects must have had at least one T₁ Gd enhancing lesion on one of the pre-treatment MRI scans; 3) Subjects must have a relapsing-remitting disease course; 4) Subjects must have had a least one documented relapse within one year prior to the screening visit (week −10); 5) Subjects must be relapse-free and not have taken corticosteroids (IV, IM and/or PO) within the 30 days prior to the screening visit; 6) Subjects may be male or female. Women of child-bearing potential must practice a medically acceptable method of birth control. Acceptable methods include oral contraceptive or double-barrier method (condom or IUD with spermicide); 7) Subjects must be between the ages of 18 and 50 years inclusive; 8) Subjects must be ambulatory, with a Kurtzke EDSS score of between 0 and 5.0 inclusive; and 9) Subjects must be willing and able to give written informed consent prior to entering the study.

Participant exclusion criteria include the following: 1) Previous use of injected glatiramer acetate; 2) Previous use of cladribine within 2 years prior to the screening visit (week −10) δ 3) Previous use of immunosuppressive agents in the last 6 months; 4) Use of experimental or investigational drugs, including I.V. immunoglobulin, within 6 months prior to study entry; 5) Use of interferon agents within 60 days prior to the screening visit; 6) Chronic corticosteroid (IV, IM and/or PO) treatment (more than 30 consecutive days) in the 6 months prior to study entry; 7) Chronic use of antioxidant substance(s) (more than 30 consecutive days) within 60 days prior to the screening visit; 8) Previous total body irradiation or total lymphoid irradiation (TLI); 9) Pregnancy or breastfeeding; 10) Significant medical or psychiatric condition that affects the subject's ability to give informed consent, or to complete the study, or any condition which the investigator feels may interfere with participation in the study (e.g. alcohol or drug abuse); 11) A known history of uncontrolled asthma; 12) A known history of sensitivity to mannitol and acetylcysteine; and 13) Inability to successfully undergo MRI scanning.

The study assessments are conducted according to the Study Task Flow Sheet below.

The study duration is 46 weeks: 10 pre-treatment weeks and 36 treatment weeks. Subjects are evaluated at study sites at weeks −10 (screening), −6, 0 (baseline), 4, 16, 28, 32 and 36 (termination). Subjects meeting all inclusion/exclusion criteria (except for MRI) at the study screening visit at week −10, return after 28±4 days for the week −6 visit. Subjects return 42±4 days after the week −6 visit for the baseline visit (week 0). The baseline MRI scan is performed 14-18 days prior to the baseline visit (week 0). Subjects meeting all inclusion/exclusion criteria and are eligible to receive the combination treatment receive their first dose of GA and NAC at the baseline visit.

Study Task Flow Sheet

| | | | Treatment Phase | | | | | | |
|---|---|---|---|---|---|---|---|---|---|
| | Pre-Treatment | | | | | | | Week 36 | |
| | Phase | | | | | | | Termination | |
| | Week −10 | | Week 0 | | | | | Early | Unsch |
| Procedure | Screening | Week −6 | Baseline | Week 4 | Week 16 | Week 28 | Week 32 | Discont. | Visit |
| Informed | X | | | | | | | | |
| Consent | | | | | | | | | |
| Signature | | | | | | | | | |
| Eligibility | X | | X^{1,2} | | | | | | |
| Criteria | | | | | | | | | |
| Medical | X | X¹ | X^{1,2} | | | | | | |
| History | | | | | | | | | |
| Historical | X | X | X^{1,2} | X | X | X | X | X | X⁹ |
| and | | | | | | | | | |
| Concomitant | | | | | | | | | |
| Medication | | | | | | | | | |
| Physical | X | | X² | | X | | | X | X⁹ |
| Examination | | | | | | | | | |
| Hearing | | | X² | X | X | X | X | X | X |
| Function | | | | | | | | | |
| Assessment | | | | | | | | | |
| Vital Signs | X | | X⁴ | X | X | X | X | X | X |
| ECG | X | | | | | | | | X⁹ |
| Chest X-ray⁵ | X | | | | | | | | |
| Neurological | X | | X² | | X | | | X | X⁹ |
| Examination | | | | | | | | | |
| Evaluation of | X | X | X² | X | X | X | X | X | X⁹ |
| Relapse⁶ | | | | | | | | | |
| First GA + NAC | | | X | | | | | | |
| Administration | | | | | | | | | |
| at Study | | | | | | | | | |
| Site | | | | | | | | | |
| Laboratory | X | | X² | X | X | | | X | X⁹ |
| Evaluation | | | | | | | | | |
| MRI | X | X | X⁷ | | | X | X | X | |
| Pregnancy | X | | X² | | | | | | |
| Test⁸ | | | | | | | | | |
| Drug | | | X³ | X | X | X | X¹⁰ | X¹⁰ | X⁹ |
| Compliance & | | | | | | | | | |
| Dispensing | | | | | | | | | |
| Adverse | | | X³ | X | X | X | X | X | X |
| Experiences | | | | | | | | | |
| Termination | | | | | | | | X | |

¹Review changes only.
²Perform prior to first study drug administration.
³Perform after study drug administration.
⁴Pre and post-dose vital signs: (30 minutes post-dose).
⁵The screening chest X-ray is deferred if a chest X-ray has been performed in the 6 months prior to screening and a report is available.
⁶Relapse evaluation are performed during scheduled as well as unscheduled visits as deemed necessary by the Investigator.
⁷Performed within 14-18 days prior to baseline visit (week 0).
⁸For women of childbearing potential. Screening: urine pregnancy test Baseline: serum pregnancy test.
⁹Assessments during an unscheduled visit are performed as deemed necessary by the investigator, except vital signs which are performed at each visit and relapse evaluation if the visit is due to subject's complaint of possible relapse.
¹⁰Study drugs are not dispensed. Only drug accountability is performed.

### Efficacy Assessment

The primary objective of the study is to evaluate the effect of N-Acetylcysteine (NAC) and Glatiramer Acetate (GA) on MRI disease activity as reflected by the total number of T₁ Gd-enhancing lesions in Relapsing-Remitting Multiple Sclerosis (RR-MS) subjects. Secondary efficacy endpoints for this study assess the treatment effect of N-Acetylcysteine (NAC) and Glatiramer Acetate (GA) on additional MRI parameters as follows: 1) Change in the sum of new T₂ lesions measured at pre-treatment (weeks −6 and 0 [baseline]) to the sum of new T₂ lesions measured in the last 8 weeks of treatment (weeks 32 and 36 [termination]); and 2) Brain Atrophy as defined by the percent of change from baseline to termination in brain volume measured according to the SIENA technique.

Tolerability is evaluated with reference to the following: 1) percentage of subjects who discontinue the study early; 2) percentage of subjects who discontinue the study early due to adverse events. Safety is evaluated with reference to 1) adverse event frequency and severity; 2) changes in vital signs and 3) blood and urine laboratory testing.

### Neurological Evaluation

Each subject is evaluated by an Examining Neurologist for the neurological exam and relapse evaluation. The Treating Neurologist sees the subject for relapse confirmation, based on the Examining Neurologists' findings, and prescribes steroid treatment if warranted. The Examining Neurologist performs all neurological evaluations throughout the study. FS, and EDSS score are assessed based on standardized neurological examination and slightly modified FS and EDSS (Neurostatus L. Kappos, Dept. of Neurology, University Hospital, CH-4031/Basel).

### Relapse Evaluation

A relapse is defined as the appearance of one or more new neurological abnormalities or the reappearance of one or more previously observed neurological abnormalities. This change in clinical state must last at least 48 hours and be immediately preceded by a relatively stable or improving neurological state of at least 30 days. This criterion is different from the clinical definition of exacerbation “at least 24 hours duration of symptoms” (Poser C M. et al. New diagnostic criteria for multiple sclerosis: Guidelines for research protocols. Ann. Neurol. 1983). Since “in study” exacerbation definition must be supported by an objective neurological evaluation (see next paragraph), a neurological deficit is sustained long enough to eliminate pseudo exacerbations.

An event is counted as a relapse only when the subject's symptoms are accompanied by observed objective neurological changes, consistent with 1) an increase of at least 0.5 in the EDSS score or one grade in the score of two or more of the seven FS; or, 2) two grades in the score of one of FS as compared to the previous evaluation. The subject is not undergoing any acute metabolic changes such as fever or other medical abnormality. A change in bowel/bladder function or in cognitive function is not entirely responsible for the changes in EDSS or FS scores.

A complete neurological assessment is performed at screening (week −10), baseline (week 0), and weeks 16 and 36 (or early termination visit). A complete neurological assessment is also performed during a schedule or unscheduled visit when the subject's complaint suggested a relapse. The decision as to whether the neurological change is considered a confirmed relapse is made by the Treating Physician, based on EDSS/FS scores assessed by the Examining Physician. Follow-up visits to monitor the course of the relapse are made at the Treating Physician's discretion, in addition to the assessment at the next scheduled visit, but the neurological assessments are performed by the Examining Physician. Subjects are instructed to telephone their study site immediately should any symptoms suggestive of a relapse appear. The subject is examined within 7 days after a symptomatic period of greater than or equal to 48 hours. The Treating Neurologist evaluates the subject once any symptom suggestive of a relapse occurs. The investigator makes the decision whether or not corticosteroids should be administered for the treatment of the relapse. A fixed dose of 1 g/day of I.V. methylprednisolone (Solumedrol®) for 3 consecutive days maximum is the treatment allowed in this protocol. No tapering off is allowed.

### Study Medication

Subjects are treated with a daily dose of 1.0 mL of COPAXONE® Injection solution for subcutaneous injection which contains 20 mg of glatiramer acetate and 40 mg of mannitol, USP in combination with a twice daily dose of 2.5 g of NAC. COPAXONE® Injection solution is supplied by Teva Neurosciences, Inc., Kansas City, Mo. NAC is supplied by Bioniche Life Science Inc., Canada.

### Results

Patients treated with COPAXONE® and N-acetylcysteine or a pharmaceutically acceptable salt thereof exhibit a reduction in the total number of T1 Gd-enhancing lesions well as a reduction of new T2 lesions. Relapsing remitting multiple sclerosis patients also exhibit a reduction in the progression of brain atrophy.

1. A method of treating a subject afflicted with a form of multiple sclerosis comprising periodically administering to the subject an amount of glatiramer acetate and an amount of N-acetylcysteine or a pharmaceutically acceptable salt thereof, wherein the amounts when taken together are effective to alleviate a symptom of the form of multiple sclerosis in the subject so as to thereby treat the subject.

2. The method of claim 1, wherein the form of multiple sclerosis is relapsing-remitting multiple sclerosis.

3. The method of claim 1, wherein the subject is a human being.

4. The method of claim 1, wherein each of the amount of glatiramer acetate when taken alone, and the amount of N-acetylcysteine or the pharmaceutically acceptable salt thereof when taken alone is effective to alleviate the symptom of the form of multiple sclerosis.

5. The method of claim 1, wherein either the amount of glatiramer acetate when taken alone, the amount of N-acetylcysteine or the pharmaceutically acceptable salt thereof when taken alone or each such amount when taken alone is not effective to alleviate the symptom of the form of multiple sclerosis.

6. The method of claim 1, wherein the symptom is the frequency of relapses, the frequency of clinical exacerbation, or the accumulation of physical disability.

7. The method of claim 1, wherein the amount of glatiramer acetate is in the range from 10 to 600 mg/week.

8. The method of claim 7, wherein the amount of glatiramer acetate is 300 mg/week.

9. The method of claim 1, wherein the amount of glatiramer acetate is in the range from 50 to 150 mg/day.

10. The method of claim 9, wherein the amount of glatiramer acetate is 100 mg/day.

11. The method of claim 1, wherein the amount of glatiramer acetate is in the range from 10 to 80 mg/day.

12. The method of claim 11, wherein the amount of glatiramer acetate is 20 mg/day.

13. The method of claim 12, wherein the glatiramer acetate is in solution with 40 mg of manitol, USP.

14. The method of claim 1, wherein the amount of N-acetylcysteine or the pharmaceutically acceptable salt thereof is in the range of 500 mg-20 g/day.

15. The method of claim 14, wherein the amount of N-acetylcysteine or the pharmaceutically acceptable salt thereof is 5 g/day.

16. The method of claim 1, wherein the periodic administration of N-acetylcysteine or the pharmaceutically acceptable salt thereof is effected twice daily at one half the amount.

17. The method of claim 1, wherein the periodic administration of glatiramer acetate is effected daily.

18. The method of claim 1, wherein the periodic administration of glatiramer acetate is effected twice daily at one half the amount.

19. The method of claim 1, wherein the periodic administration of glatiramer acetate is effected once every 5 to 9 days.

20. The method of claim 1, wherein the periodic administration of N-acetylcysteine or the pharmaceutically acceptable salt thereof is effected twice daily.

21. The method of claim 1, wherein the administration of the glatiramer acetate substantially precedes the administration of the N-acetylcysteine or the pharmaceutically acceptable salt thereof.

22. The method of claim 1, wherein the administration of the N-acetylcysteine or the pharmaceutically acceptable salt thereof substantially precedes the administration of the glatiramer acetate.

23. The method of claim 1, wherein the administration of the glatiramer acetate is effected subcutaneously, intraperitoneally, intravenously, intramuscularly, intraocularly or orally and the administration of the N-acetylcysteine or the pharmaceutically acceptable salt thereof is effected orally.

24. The method of claim 23, wherein the administration of the glatiramer acetate is effected subcutaneously and the administration of the N-acetylcysteine or the pharmaceutically acceptable salt thereof is effected orally.

25. A package comprising
a. a first pharmaceutical composition comprising an amount of glatiramer acetate and a pharmaceutically acceptable carrier;
b. a second pharmaceutical composition comprising an amount of N-acetylcysteine or a pharmaceutically acceptable salt thereof and a pharmaceutically acceptable carrier; and
c. instructions for use of the first and second pharmaceutical compositions together to alleviate a symptom of a form of multiple sclerosis in a subject.

26-37. (canceled)
